(19) United States
(12) Patent Application Publication
(10) Pub. No.: US 2024/0207159 A1
(43) Pub. Date: Jun. 27, 2024

(54) SKIN PENETRATION IMPROVER

(71) Applicant: KANEKA CORPORATION, Osaka (JP)

(72) Inventors: Tadao Tsuji, Osaka (JP); Satohiro Yanagisawa, Tokyo (JP)

(73) Assignee: KANEKA CORPORATION, Osaka (JP)

(21) Appl. No.: 18/589,817

(22) Filed: Feb. 28, 2024

Related U.S. Application Data
(63) Continuation of application No. PCT/JP2022/031629, filed on Aug. 22, 2022.

(30) Foreign Application Priority Data
Sep. 3, 2021 (JP) 2021-144091

(51) Int. Cl. A61K 8/55; A61K 8/64; A61Q 19/00

(52) U.S. Cl. CPC A61K 8/553; A61K 8/64; A61Q 19/00

(57) ABSTRACT

Provided are a skin penetration improver to improve a skin penetration property of a hydrophilic compound, and a cosmetic product and an external preparation including the skin penetration improver and a hydrophilic compound. The skin penetration improver is characterized by including a lipopeptide biosurfactant and a phospholipid, and the skin penetration improver improves a skin penetration property of a hydrophilic compound. The cosmetic product and the external preparation are characterized by including the skin penetration improver and a hydrophilic compound.

### TECHNICAL FIELD

One or more embodiments of the present invention relate to a skin penetration improver to improve a skin penetration property of a hydrophilic compound, and a cosmetic product and an external preparation comprising the skin penetration improver and a hydrophilic compound.

### BACKGROUND

A drug may be delivered at least to the epidermis in order to improve the condition of skin and a skin disease. In addition, a transdermal formulation has the advantages of reduced burden on the liver, reduced side effects such as gastrointestinal tract disturbance, simplicity of use and persistence of action unlike an oral drug. On the one hand, a drug has to be delivered to a deeper dermal tissue than epidermis from a transdermal formulation in order to exert the effect of the active ingredient in the transdermal formulation, since blood capillaries exist in deeper dermal tissue than an epidermal tissue.

The skin structure is divided into epidermis, dermis and a subcutaneous tissue from the outermost layer, and epidermis is further divided into stratum corneum, stratum granulosum, stratum spinosum and stratum basale. While the stratum basale to the stratum granulosum are composed of living cells, the stratum corneum is composed of dead cells. The space therebetween is filled with lipids such as ceramide and cholesterol and is thus lipophilic. As a result, the stratum corneum functions as a biological barrier to protect against external stimuli and to retain moisture in the body. A hydrophilic drug is therefore generally unsuitable as an active ingredient of a transdermal formulation, since the drug is difficult to penetrate the stratum corneum.

Techniques to mix a lipophilic component in a formulation and to capsulate a hydrophilic compound in a microcapsule have been developed in order to improve a skin penetration property of a hydrophilic compound. For example, Patent document 1 discloses an anhydrous composition that contains a water-soluble particle, an organic lipophilic gelator such as a styrene block copolymer and oil and that is used for a keratin substance such as skin. Patent document 2 discloses a mixed micelle formed from the specific phosphatide and a cholanate salt or dihexanoyllecithin. Patent document 3 discloses a colloidal nanocarrier for a hydrophilic active substance, in which inverse emulsion is formed. Patent document 4 discloses a liposome formulation containing a phospholipid component and polyglycerol in addition to an active ingredient.

In addition, Patent document 4 discloses a micro needle sheet to improve or accelerate the skin penetration of an N-acylaminoamide inhibitor. But a micro needle sheet is not preferred, since it physically damages a skin.

### PATENT DOCUMENTS

Patent document 1: JP 2021-104934 A
Patent document 2: JP H8-245339 A
Patent document 3: JP 2013-530168 A
Patent document 4: JP 2019-69909 A

Various technologies to improve the skin penetration property of a hydrophilic compound have been developed as described above. In addition, a technology to surely deliver a hydrophilic compound to not only the stratum corneum and an epidermal tissue but also a dermal tissue in which blood capillaries exist has been demanded.

Thus, one or more embodiments of the present invention provide a skin penetration improver to improve the skin penetration property of a hydrophilic compound, and a cosmetic product and an external preparation comprising the skin penetration improver and a hydrophilic compound.

### SUMMARY

The inventors of one or more embodiments of the present invention repeated intensive studies in order to solve the above. As a result, the inventors completed one or more embodiments of the present invention by finding that the skin penetration property of a hydrophilic compound can be remarkably improved by using a lipopeptide biosurfactant and a phospholipid in combination.

One or more embodiments of the present invention are hereinafter described.

[1] A skin penetration improver,
comprising a lipopeptide biosurfactant and a phospholipid,
wherein the skin penetration improver improves a skin penetration property of a hydrophilic compound.

[2] The skin penetration improver according to the above [1], wherein a mass ratio of the lipopeptide biosurfactant to the phospholipid is 0.5 times or more and 3 times or less.

[3] The skin penetration improver according to the above [1] or [2], wherein the lipopeptide biosurfactant is surfactin or a salt thereof represented by the following formula (I):

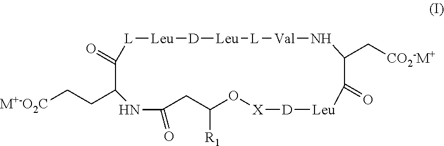

wherein
X is an amino acid residue selected from leucine, isoleucine and valine,
R¹ is a C₉₋₁₈ alkyl group,
M⁺ is an alkali metal ion or a quaternary ammonium ion.

[4] The skin penetration improver according to any one of the above [1] to [3], wherein the phospholipid is a glycerophospholipid represented by the following formula (II):

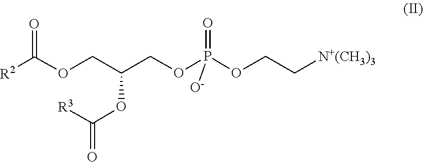

wherein R² and R³ are independently a C₁₀₋₂₄ alkyl group or a C₁₀₋₂₄ alkenyl group.

[5] A cosmetic product comprising the skin penetration improver according to any one of the above [1] to [4] and a hydrophilic compound.

[6] An external preparation comprising the skin penetration improver according to any one of the above [1] to [4] and a hydrophilic compound.

[7] Use of a composition for improving a skin penetration property of a hydrophilic compound, wherein the composition comprises a lipopeptide biosurfactant and a phospholipid.

[8] The use according to the above [7], wherein a mass ratio of the lipopeptide biosurfactant to the phospholipid is 0.5 times or more and 3 times or less.

[9] The use according to the above [7] or [8], wherein the lipopeptide biosurfactant is surfactin or a salt thereof represented by the above formula (I).

[10] The use according to any one of the above [7] to [9], wherein the phospholipid is a glycerophospholipid represented by the above formula (II).

[11] A method for improving a skin penetration property of a hydrophilic compound, comprising the step of mixing a lipopeptide biosurfactant and a phospholipid in a composition comprising the hydrophilic compound.

[12] The method according to the above [11], wherein a mass ratio of the lipopeptide biosurfactant to the phospholipid is 0.5 times or more and 3 times or less.

[13] The method according to the above or [12], wherein the lipopeptide biosurfactant is surfactin or a salt thereof represented by the above formula (I).

[14] The method according to any one of the above to [13], wherein the phospholipid is a glycerophospholipid represented by the above formula (II).

A compound that is water-miscible solvent but more lipophilic than water, such as ethanol and isopropanol, has been conventionally mixed in cosmetics, an external preparation or the like in order to improve the skin penetration property of a hydrophilic compound. Ethanol and isopropanol, however, wreak havoc on the skin. On the one hand, a lipopeptide biosurfactant and a phospholipid that are essential components of the skin penetration improver according to one or more embodiments of the present invention may be harmless to the skin or have very low toxicity, since a lipopeptide biosurfactant is a peptide and a phospholipid is a structural component of a cell wall. In addition, the inventors experimentally found that the skin penetration improver of one or more embodiments of the present invention can deliver a hydrophilic compound to a dermal tissue having blood capillaries. Accordingly, the skin penetration improver of one or more embodiments of the present invention is industrially very useful as a safe constituent component to improve the skin penetration property of a hydrophilic compound contained in cosmetics and an external preparation.

### BRIEF DESCRIPTION OF THE DRAWINGS

The patent or application file contains at least one drawing executed in color. Copies of this patent or patent application publication with color drawing (s) will be provided by the office upon request and payment of the necessary fee.

FIG. 1 is an HE stain photograph of the experimentally used human skin sample.

FIGS. 2A-2D are magnified cross-section photographs of skin samples to show the skin penetration property of the water-soluble fluorescent dye in (1) the water-soluble fluorescent dye solution (FIG. 2A), (2) the solution of the water-soluble fluorescent dye and surfactin sodium salt (FIG. 2B), (3) the solution of the water-soluble fluorescent dye and the phospholipid (FIG. 2C), and (4) the solution of the water-soluble fluorescent dye, surfactin sodium salt and the phospholipid (FIG. 2D).

### DETAILED DESCRIPTION OF THE EMBODIMENTS

The skin penetration improver of one or more embodiments of the present invention comprises a lipopeptide biosurfactant and a phospholipid. One or more embodiments of the present invention are hereinafter described in more detail and is not restricted to the following specific examples. For example, even if 2 or more embodiments are hereinafter separately described, such embodiments can be combined and such a combination is included in the range of one or more embodiments of the present invention.

The lipopeptide biosurfactant is a natural surfactant having a hydrophilic cyclic peptide part and a non-hydrophilic long chain hydrocarbon group. The cyclic peptide part contains 1 or more anionic groups such as a carboxy group and a phenolic hydroxy group. The skin penetration property of a hydrophobic compound can be improved by one or more embodiments of the present invention by combining a phospholipid with a lipopeptide biosurfactant, though the action mechanism thereof is not clear.

An example of the lipopeptide biosurfactant includes one or more lipopeptide biosurfactants selected from surfactin, arthrofactin, iturin and salts thereof, and the lipopeptide biosurfactant may be surfactin or a salt thereof.

The surfactin salt is a compound represented by the general formula (I) or a composition comprising 2 or more of the compounds.

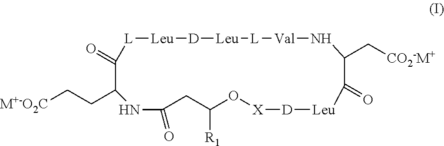

wherein X is an amino acid residue selected from leucine, isoleucine and valine; R¹ is a C₉₋₁₈ alkyl group; M⁺ is an alkali metal ion or a quaternary ammonium ion.

The amino acid residue as the ‘X’ may be either in an L-form or a D-form, and the L-form is preferred.

The “C₉₋₁₈ alkyl group” means a linear or branched monovalent saturated hydrocarbon group having a carbon number of 9 or more and 18 or less. An example of the C₉₋₁₈ alkyl group includes n-nonyl, 6-methyloctyl, 7-methyloctyl, n-decyl, 8-methylnonyl, n-undecyl, 9-methyldecyl, n-dodecyl, 10-methylundecyl, n-tridecyl, 11-methyldodecyl, n-tetradecyl, n-pentadecyl, n-hexadecyl, n-heptadecyl and n-octadecyl.

The alkali metal ion is not particularly restricted, is exemplified by a lithium ion, a sodium ion and a potassium ion, and may be a sodium ion.

An example of a substituent of the quaternary ammonium ion includes an organic group exemplified by an alkyl group such as methyl, ethyl, n-propyl, isopropyl, n-butyl and tert-butyl; an aralkyl group such as benzyl, methylbenzyl and phenylethyl; and an aryl group such as phenyl, toluyl and xylyl. An example of the quaternary ammonium ion includes a tetramethylammonium ion, a tetraethylammonium ion and a pyridinium ion.

Arthrofactin is represented by the general formula (III).

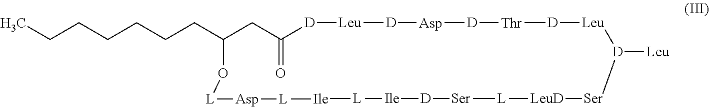

Arthrofactin has one D-aspartic acid and one L-aspartic acid respectively in the structure, which may form a salt with an alkali metal ion or a quaternary ammonium ion.

Iturin is represented by the general formula (IV).

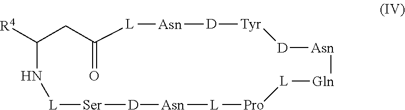

In the formula (IV), R⁴ is a C₉₋₁₈ alkyl group such as —(CH₂)₁₀CH₃, —(CH₂)₈CH(CH₃)CH₂CH₃ and —(CH₂)₉CH(CH₃)₂.

One kind of or two or more kinds of the lipopeptide biosurfactants may be used. For example, a mixture of two or more kinds of surfactin salts of which R¹ are different from each other may be used as the lipopeptide biosurfactant. The lipopeptide biosurfactant can be isolated from a culture medium prepared by cultivating a microorganism that produces the target lipopeptide biosurfactant in accordance with a known method. The lipopeptide biosurfactant may be a purified product or an unpurified product. An example of such an unpurified product includes the culture medium as it is. An example of a microorganism that produces surfactin includes a strain belonging to Bacillus subtilis. The lipopeptide biosurfactant produced by a chemical synthesis method may be similarly used.

A phospholipid means one of complex lipids and a general term for lipids having a phosphate ester or a phosphonate ester in the molecule. A phospholipid is a main component of the membrane of an organelle such as endoplasmic reticulum, mitochondria, Golgi body, lysosome and chloroplast in addition to a plasma membrane and a nuclear membrane that constitute a cell, and forms the biomembranes as a bilayer. A phospholipid is classified into a glycerophospholipid containing glycerin as a common structural component and a sphingophospholipid containing sphingosine, which is an aminoalcohol having the carbon number of 18, as a common structural component. In addition, an example of a phospholipid includes lysolecithin produced by treating a glycerophospholipid or a sphingophospholipid with phospholipase A2 to remove one of the hydrophobic tail parts.

A glycerophospholipid is a phospholipid having the structure formed by combining two fatty acids, glycerin, phosphate and choline, and for example, is represented by the following formula (II):

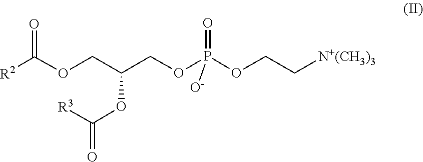

wherein R² and R³ are independently a C₁₀₋₂₄ alkyl group or a C₁₀₋₂₄ alkenyl group.

In the above-described formula (II), an example of the C₁₀₋₂₄ alkyl group includes n-decyl, 8-methylnonyl, n-undecyl, 9-methyldecyl, n-dodecyl, 10-methylundecyl, n-tridecyl, 11-methyldodecyl, n-tetradecyl, n-pentadecyl, n-hexadecyl, n-heptadecyl, n-octadecyl, n-nonadecyl, n-icosyl, n-docosyl and n-tetracosyl. An example of the C₁₀₋₂₄ alkenyl group includes decenyl, dodecenyl, tetradecenyl, hexadecenyl, octadecenyl, icocenyl, docosenyl and tetracosenyl.

An example of the glycerophospholipid includes dioleoylphosphatidylcholine (DOPC), dilauroylphosphatidylcholine (DLPC), dimyristoylphosphatidylcholine (DMPC), dipalmitoylphosphatidylcholine (DPPC), distearoylphosphatidylcholine (DSPC), 1-palmitoyl-2-oleoylphosphatidylcholine (POPC), 1-stearoyl-2-myristoylphosphatidylcholine and dilinoleoylphosphatidylcholine. A phosphatidylcholine is also referred to as lecithin. A lecithin derived from egg yolk or soy, and a derivative thereof, such as hydrogenated lecithin, may be also used.

A sphingophospholipid has a ceramide formed by binding a fatty acid as the R⁵ part to a sphingoid through an amide bond as a common structure and contains a phosphate and a base in the R⁶ part. For example, a sphingophospholipid is represented by the following formula (V):

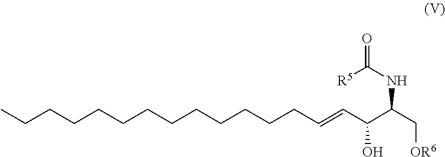

wherein R⁵ is a C₁₀₋₂₄ alkyl group or a C₁₀₋₂₄ alkenyl group, and R⁶ is a phosphocholine group (—P(═O)(—O—)—OCH₂CH₂N⁺ (—CH₃)₃) or a phosphoethanolamine group (—P(═O)(—O—)—OCH₂CH₂NH₂).

Examples of the C₁₀₋₂₄ alkyl group and the C₁₀₋₂₄ alkenyl group in the formula (V) include the same groups as the C₁₀₋₂₄ alkyl group and the C₁₀₋₂₄ alkenyl group in the formula (II).

A lysolecithin is a single strand phospholipid, which has only one hydrophobic tail part, while a main phospholipid such as a glycerophospholipid and a sphingophospholipid is a double strand phospholipid, which has two hydrophobic tail parts. For example, lysolecithin is produced by treating a glycerophospholipid or a sphingophospholipid with phospholipase A2 to remove one of the hydrophobic tail parts.

An example of a lysolecithin includes lysophosphatidylcholine, lysophosphatidic acid, sphingosine-1-phosphate and sphingosylphosphorylcholine. Lysophosphatidylcholine has the structure produced by removing the fatty acid at the 2-position of a phosphatidylcholine with phospholipase A2, and lysophosphatidic acid has the structure produced by removing the choline from a lysophosphatidylcholine.

The skin penetration improver of one or more embodiments of the present invention comprises the lipopeptide biosurfactant and the phospholipid. The ratio of the components may be appropriately adjusted to the range to improve the skin penetration property of a hydrophilic compound. For example, a mass ratio of the lipopeptide biosurfactant to the phospholipid may be adjusted to 0.5 times or more and 3 times or less. The ratio may be 1 time or more, 1.2 times or more, or 1.5 times or more, and 2.5 times or less, or 2 times or less.

The usage amounts of the lipopeptide biosurfactant and the phospholipid may be also appropriately adjusted to the range to improve the skin penetration property of a hydrophilic compound. In general, since a hydrophilic compound as an active ingredient is relatively expensive, the lipopeptide biosurfactant and the phospholipid may be excessively used in comparison with a hydrophilic compound as an active ingredient. For example, the total mass ratio of the lipopeptide biosurfactant and the phospholipid to a hydrophilic compound may be adjusted to 1 time or more and 100 times or less. The mass ratio may be adjusted to 50 times or less or 10 times or less.

The total amount of the lipopeptide biosurfactant and the phospholipid in the composition containing a hydrophilic compound, the lipopeptide biosurfactant and the phospholipid is larger, the skin penetration property of a hydrophilic compound may be improved. The total concentration of the lipopeptide biosurfactant and the phospholipid in the above-described composition may be adjusted to, for example, 0.01 mass % or more and 5 mass % or less. The total concentration may be 0.05 mass % or more, or 0.1 mass % or more, and 3 mass % or less, 2 mass % or less, or 1 mass % or less.

The skin penetration improver of one or more embodiments of the present invention can improve the skin penetration property of a hydrophilic compound, which is generally hardly permeant and permeable into the stratum corneum, and deliver a hydrophilic compound to at least the dermis. Such a hydrophilic compound is not particularly restricted as long as the hydrophilic compound has an affinity for a water molecule and for example, means a compound that is dissolved within 30 minutes when 1 g or 1 mL of the compound is added to less than 30 mL of water and the mixture is strongly shaken at 20±5° C. for 30 seconds every 5 minutes.

The hydrophilic compound of which skin penetration property should be improved by one or more embodiments of the present invention is not particularly restricted as long as the compound is hydrophilic and should be permeant into the skin, and an example of the hydrophilic compound includes a skin-lightening agent, an antioxidant agent, a moisturizer, a vitamin, an amino acid, a hair grower, an antimicrobial agent, a hormonal agent and an enzyme.

An example of a skin-lightening agent includes L-ascorbic acid and a derivative thereof, a pantothenic acid derivative, tranexamic acid and a derivative thereof, a salicylic acid derivative such as potassium 4-methoxysalicylate, a phenol derivative, a placenta extract, and a botanical extract such as a chamomilla recutita extract.

An example of L-ascorbic acid and a derivative thereof includes L-ascorbic acid, L-ascorbic acid phosphate ester, L-ascorbic acid 2-sulfate ester, L-ascorbic acid 2-glucoside, or salts thereof; an L-ascorbate monoalkyl ester such as L-ascorbyl monostearate, L-ascorbyl monopalmitate and L-ascorbyl monooleate; an L-ascorbyl monoester such as L-ascorbyl monophosphate ester and L-ascorbyl 2-sulfate ester; an L-ascorbate dialkyl ester such as L-ascorbyl diphosphate ester; an L-ascorbyl trialkyl ester such as L-ascorbyl tristearate, L-ascorbyl tripalmitate and L-ascorbyl trioleate; an L-ascorbyl triester such as L-ascorbyl triphosphate ester; and an L-ascorbyl glucoside such as L-ascorbyl 2-glucoside.

An example of a pantothenic acid derivative includes pantothenyl ethyl ether and panthenol.

An example of a tranexamic acid derivative includes a tranexamic acid dimer such as trans-4-(trans-aminomethylcyclohexanecarbonyl)aminomethylcyclohexanecarboxyli c acid hydrochloride; an ester of tranexamic acid and hydroquinone, such as trans-4-aminomethyl-1-cyclohexanecarboxylic acid 4′-hydroxyphenyl ester; an ester of tranexamic acid and gentisic acid, such as 2-(trans-4-aminomethylcyclohexylcarbonyloxy)-5-hydroxybenzoic acid and a salt thereof; and an amide of tranexamic acid, such as trans-4-aminomethylcyclohexanecarboxylic acid methylamide and a salt thereof, trans-4-(P-methoxybenzoyl)aminomethylcyclohexanecarboxylic acid and a salt thereof, and trans-4-guanidinomethylcyclohexanecarboxylic acid and a salt thereof.

An example of an antioxidant agent includes glycyrrhizin, a glycyrrhizinate salt, allantoin, thiotaurine, glutathione, catechin, albumin, ferritin and metallothionein. An example of a glycyrrhizinate salt includes dipotassium glycyrrhizinate and ammonium glycyrrhizinate.

An example of a moisturizer includes urea, glycerin, xylitol and erythritol.

An example of a vitamin includes a hydrophilized vitamin A derivative; vitamin B6 and a vitamin B6 derivative such as vitamin B6 hydrochloride; a nicotinic acid and a nicotinic acid derivative such as nicotinamide; a hydrophilized vitamin E derivative; and hydrophilized β-carotene.

An example of an amino acid includes hydroxyproline, serine, trimethylglycine and arginine.

An example of a hair grower includes pantothenyl ethyl ether, adenosine, β-glycyrrhetinic acid and minoxidil.

An example of an antimicrobial agent includes resorcin, sulfur and salicylic acid.

An example of a hormonal agent includes oxytocin, corticotropin, vasopressin, secretin, gastrin, calcitonin, hinokitiol and ethinyl estradiol.

An example of an enzyme includes trypsin, lysozyme chloride, chymotrypsin, semi-alkaline proteinase, serrapeptase, lipase and hyaluronidase.

The concentration of a hydrophilic compound in the composition comprising the hydrophilic compound, the lipopeptide biosurfactant and the phospholipid may be appropriately adjusted depending on the kind and the property of the hydrophilic compound and may be adjusted to, for example, 0.01 mass % or more and 10 mass % or less. The concentration may be 0.1 mass % or more and 5 mass % or less.

The skin penetration improver of one or more embodiments of the present invention can be used for a cosmetic product, an external preparation or the like. Such a cosmetic product and an external preparation may comprise other additive that commonly used for a cosmetic product and an external preparation depending on a dosage form thereof in addition to a hydrophilic compound, the lipopeptide biosurfactant and the phospholipid. An example of such an additive includes a solvent such as water and glycerin, moisturizer, water retention agent, wetting agent, skin barrier, oral hygienic agent, fragrance, ultraviolet absorber, antioxidant, emollient agent, solubilizer, anti-inflammatory drug, humectant, preservative, fungicide, pigment, perfume and powders.

The skin penetration improver of one or more embodiments of the present invention can be easily produced by mixing the lipopeptide biosurfactant and the phospholipid in a solvent. The cosmetic product and the external preparation comprising the skin penetration improver of one or more embodiments of the present invention can be produced by mixing a hydrophilic compound, the lipopeptide biosurfactant and the phospholipid in a solvent, or mixing the lipopeptide biosurfactant and the phospholipid or the solution thereof into a solution or a suspension of a hydrophilic compound, or adding and mixing a solution of the lipopeptide biosurfactant and the phospholipid into a solid composition of a hydrophilic compound. The above-described other additive may be appropriately added and mixed depending on each dosage form.

The solvent may be an aqueous solvent, since one or more embodiments of the present invention are to improve the skin penetration property of a hydrophilic compound. The term “aqueous solvent” in this disclosure means water or a mixed solvent of a water miscible organic solvent and water. The water miscible organic solvent means an organic solvent that can mix with water without restriction, is exemplified by a C₁₋₃ alcohol and may be ethanol or isopropanol. A ratio of a water miscible organic solvent in the above-described mixed solvent may be 10 mass % or less, 5 mass % or less, or 2 mass % or less. The aqueous solvent may be a buffer solution. The pH of such a buffer solution is not particularly restricted and may be 5.0 or more and 13.0 or less.

The skin penetration property of a hydrophilic compound becomes improved by the coexistence of the hydrophilic compound, the lipopeptide biosurfactant and the phospholipid in the presence of a solvent in any kind of way according to one or more embodiments of the present invention; as a result, it becomes possible to exert the effect of the hydrophilic compound on the skin and the whole body with reducing the burden on the liver and a gastrointestinal disorder or the like.

The present application claims the benefit of the priority date of Japanese patent application No. 2021-144091 filed on Sep. 3, 2021. All of the contents of the Japanese patent application No. 2021-144091 filed on Sep. 3, 2021, are incorporated by reference herein.

### EXAMPLES

Hereinafter, the examples are described to demonstrate one or more embodiments of the present invention more specifically, but one or more embodiments of the present invention are in no way restricted by the examples, and the examples can be appropriately modified to be carried out within a range that adapts to the contents of this specification. Such a modified example is also included in the range of one or more embodiments of the present invention.

### Example 1: Transdermal Absorption Test

A circle human skin sample having a thickness of about 700 μm was attached to a test cell of an In vitro transdermal penetration test device (“TransView C12” manufactured by CosMED). The test cell was an open system Franz diffusion cell having an effective diameter of 1.7 cm, an area of 2.27 cm² and a receptor liquid volume of 5.75 mL. An electrical resistance value was measured to confirm that there was not any problems with the sample. Then, the test cell was filled with a phosphate buffer solution of pH 7.0 as a receptor liquid. The phosphate buffer solution of pH 7.01 (500 μL) containing 1.5 mass % surfactin sodium salt (SF), 1 mass % phospholipid (DMPC: 1,2-dimyristoyl-sn-glycero-3-phosphocholine) and 10 mM water-soluble fluorescent dye (calcein) was applied to the skin sample, and the skin sample was installed at the test device of which temperature was maintained at 32° ° C. to start the test.

In addition, similar tests were conducted for comparison using a PBS solution of pH 7.09 containing 10 mM water-soluble fluorescent dye, a PBS solution of pH 6.99 containing 10 mM water-soluble fluorescent dye+1.5 mass % SF, and a PBS solution of pH 7.08 containing 10 mM water-soluble fluorescent dye+1 mass % phospholipid.

The skin sample was cut 6 hours after the start of the test, embedded in O. C. T. Compound, cooled using cooled acetone to be frozen, and sliced to prepare a frozen section. The cut surface was observed using a fluorescence microscope. Separately, the used human skin sample was dyed using hematoxylin-eosin and observed under magnification.

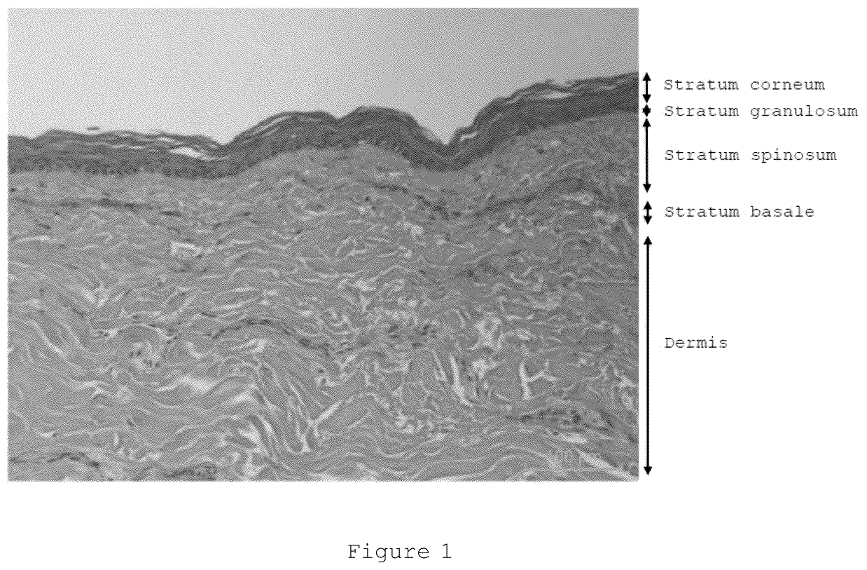

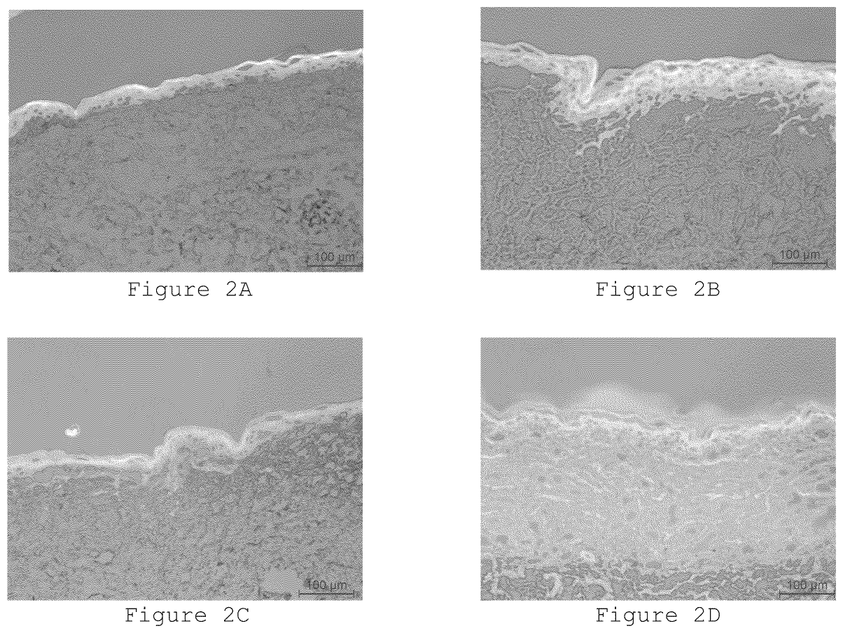

The result of the human skin sample is shown in FIG. 1, the result of the 10 mM water-soluble fluorescent dye PBS solution is shown in FIG. 2A, the result of the 10 mM water-soluble fluorescent dye+1.5 mass % SF PBS solution is shown in FIG. 2B, the result of the 10 mM water-soluble fluorescent dye+1 mass % phospholipid PBS solution is shown in FIG. 2C, and the result of the 10 mM water-soluble fluorescent dye+1 mass % phospholipid+1.5 mass % SF PBS solution is shown in FIG. 2D.

The PBS solution of the water-soluble fluorescent dye remained on the surface and could not infiltrate the deeper area than the stratum corneum as shown in FIG. 2 (FIG. 2A).

When surfactin or a phospholipid was added, the water-soluble fluorescent dye reached deeper epidermal tissue than the stratum corneum but mainly remained in the stratum corneum (FIGS. 2B-2C). The thickness of the epidermis is about 100 μm.

On the one hand, when both of surfactin and a phospholipid were added, the water-soluble fluorescent dye clearly infiltrated the epidermal tissue and also reached the dermis (FIG. 2D).

It was found from the above-described results that the skin penetration property of a water-soluble compound can be remarkably improved by the combination of surfactin and a phospholipid.

### Example 2: Transdermal Absorption Test Using Cosmetics Component

The skin penetration properties of niacinamide and arbutin, which are often mixed in cosmetics as active ingredients, were evaluated by a similar test to Example 1. A circle human skin sample having a thickness of about 700 μm was attached to a test cell of an In vitro transdermal penetration test device (“TransView C12” manufactured by CosMED). The test cell is an open system Franz diffusion cell having an effective diameter of 2.0 cm, an area of 3.14 cm- and a receptor liquid volume of 2.4 mL. An electrical resistance value was measured to confirm that there were not any problems with the sample. Then, the test cell was filled with a phosphate buffer solution of pH 7.0 as a receptor liquid. The phosphate buffer solution of pH 7.0 containing 2 mass % surfactin sodium salt (SF), 1 mass % natural lecithin and 5 mass % active ingredient was applied to the skin sample at a rate of 2 mg/cm², and the skin sample was installed at the test device of which temperature was maintained at 32° C. to start the test. A phosphate buffer solution containing 5 mass % active ingredient was concurrently subjected to the test as control.

The skin sample was taken out of the test cell 6 hours after the start of the test, and the surface was wiped using cotton. Then, the first round of tape stripping, hereinafter referred to as “TS”, was conducted using a dedicated tape, hereinafter referred to as “tape”. Specifically, the tape was attached to the skin sample and then peeled. A mixed solvent (15 mL) of ethanol:water=1:1 by v/v was added thereto, and ultrasonic wave was applied to the mixture for 1 hour for extraction. The obtained liquid was retrieved, and the above-described mixed solvent was added thereto to adjust the total amount to 20 mL as an analytic sample of “wiping+first layer”.

Then, the second or later round of TS were conducted. The number of the total times of TS was 15 at a maximum, and the number of times was adjusted depending on the condition of the skin sample surface. The tapes of second or later round were combined, and the extraction using aqueous ethanol and the total amount adjustment were conducted in a similar condition to the above to prepare an analytic sample of “penetration amount into stratum corneum”.

In addition, the remaining skin sample was finely cut and divided into two tubes for crushing. Water (0.5 mL) was added into each tube, and the skin was crushed using a crushing machine for 20 minutes. The liquid containing the crushed skin was retrieved from two tubes into one centrifuge test. Then, ethanol (12 mL) was added thereto, and the mixture was efficiently stirred and centrifuged to obtain a supernatant. Water was added to the obtained supernatant to adjust the total amount to 20 mL to prepare an analytic sample of “penetration amount into epidermis/dermis”.

The receptor liquid was also retrieved, and water was added thereto to adjust the total amount to 20 mL to prepare an analytic sample of “passing amount”. The amounts of each active ingredient in each obtained solution were measured by HPLC.

The amounts of the active ingredients retrieved from each sample of the above-described “wiping+first layer”, “penetration amount into stratum corneum”, “penetration amount into epidermis/dermis” are shown in Table 1 as ratios to 100 as the total amount retrieved from 4 samples.

TABLE 1

| | Active ingredient | | | |
|---|---|---|---|---|
| | Niacinamide | | Arbutin | |
| Test section | Control | Example | Control | Example |
| Wiping + first layer (%) | 89 | 24 | 95 | 52 |
| Penetration amount | 7 | 39 | 5 | 25 |
| into stratum corneum (%) | | | | |
| Penetration amount into | 5 | 37 | 0 | 23 |
| epidermis/dermis (%) | | | | |
| Passing amount (%) | 0 | 0 | 0 | 0 |
| Total retrieved amount (%) | 100 | 100 | 100 | 100 |

It was confirmed from the results shown in Table 1 that the skin penetration property into the skin of both of niacinamide and arbutin can be remarkably improved by the combination of surfactin and a phospholipid in comparison with the aqueous solution. The total amount of the active ingredient retrieved from the 4 samples nearly corresponded to the active ingredient amount applied to the skin sample at the start of the test in each test.

### Example 3: Effect of Concentrations Phospholipid and Surfactin

The effect of the concentrations of phospholipid and a surfactin on the penetration of an active ingredient was tested in a similar condition to Example 2 using niacinamide as an active ingredient. The condition of Example 2 was used as a benchmark of the concentrations of a phospholipid and surfactin in the samples applied to the skin, and the concentration, the ½ concentration thereof and the 1/10 concentration thereof were tested. All of the concentrations of niacinamide in the samples were adjusted to 5 mass %. The ratio of the active ingredient amounts in each sample to 100 of the total retrieved amount are shown.

TABLE 2

| | Control | Example | ½ conc. | 1/10 conc. |
|---|---|---|---|---|
| Wiping + first layer (%) | 65 | 19 | 44 | 41 |
| Penetration amount | 11 | 23 | 23 | 26 |
| into stratum corneum (%) | | | | |
| Penetration amount into | 8 | 18 | 18 | 18 |
| epidermis/dermis (%) | | | | |
| Passing amount (%) | 15 | 39 | 15 | 15 |
| Total retrieved amount (%) | 100 | 100 | 100 | 100 |

It was confirmed from the results shown in Table 2 that the effect to accelerate the penetration of an active ingredient into the dermis layer can be sufficiently improved, even if the concentrations of a phospholipid and surfactin are adjusted to ½ or 1/10.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

1. A method for improving a skin penetration property of a hydrophilic compound, comprising the step of mixing a lipopeptide biosurfactant and a phospholipid in a composition comprising the hydrophilic compound,
wherein the hydrophilic compound is delivered to a dermal tissue.

2. The method according to claim 1, wherein a mass ratio of the lipopeptide biosurfactant to the phospholipid is 0.5 times or more and 3 times or less.

3. The method according to claim 1, wherein the lipopeptide biosurfactant is surfactin or a salt thereof represented by the following formula (I): 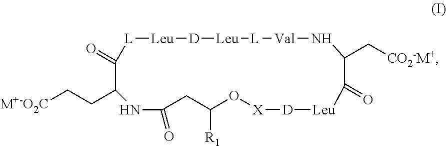
wherein:
X is an amino acid residue selected from leucine, isoleucine and valine,
R¹ is a C₉₋₁₈ alkyl group, and
M⁺ is an alkali metal ion or a quaternary ammonium ion.

4. The method according to claim 1, wherein the phospholipid is a glycerophospholipid represented by the following formula (II): 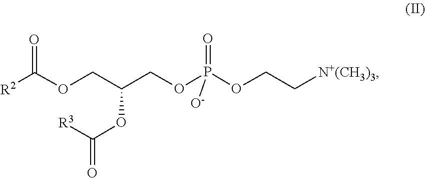
wherein R² and R³ are independently a C₁₀₋₂₄ alkyl group or a C₁₀₋₂₄ alkenyl group.

5. The method according to claim 1, wherein the composition is a cosmetic product.

6. The method according to claim 1, wherein the composition is an external preparation.
